(19) United States
(12) Patent Application Publication
(10) Pub. No.: US 2013/0338460 A1
(43) Pub. Date: Dec. 19, 2013

(54) Wearable Device for Continuous Cardiac Monitoring

(71) Applicants: David Da He, Cambridge, MA (US); Charles G. Sodini, Belmont, MA (US); Eric Steven Winokur, Danvers, MA (US)

(72) Inventors: David Da He, Cambridge, MA (US); Charles G. Sodini, Belmont, MA (US); Eric Steven Winokur, Danvers, MA (US)

(21) Appl. No.: 13/803,165

(22) Filed: Mar. 14, 2013

Related U.S. Application Data
(60) Provisional application No. 61/660,987, filed on Jun. 18, 2012.

(51) Int. Cl. A61B 5/0205; A61B 5/00; A61B 5/04; A61B 5/1455; A61B 5/0428; A61B 5/029; A61B 5/026

(52) U.S. Cl. CPC A61B 5/0205; A61B 5/029; A61B 5/0004; A61B 5/0022; A61B 5/6824; A61B 5/6823; A61B 5/6829; A61B 5/0261; A61B 5/14551; A61B 5/0428; A61B 5/04012; A61B 5/486
USPC 600/324; 600/484; 600/479; 600/480; 600/500

(57) ABSTRACT

A physiological monitor for measuring a pulsatile motion signal (MoCG) that is delayed from, but at the same rate as, the heartbeat of a user. In one embodiment, the system includes a housing configured to be worn on the body of a user; at least one MoCG sensor, within the housing, that measures a pulsatile motion signal (MoCG) that is delayed from, but at the same rate as, the heartbeat of the user; and at least one data processor that calculates, solely based on an output of the at least one MoCG sensor, at least one of (i) heart rate (HR) and activity level for the user, and (ii) respiratory rate (RR), stroke volume (SV), and cardiac output (CO) for the user. In another embodiment, the at least one data processor is within the housing.

### RELATED APPLICATIONS

This application claims priority to U.S. Provisional Application 61/660,987 filed Jun. 18, 2012, which is herein incorporated by reference.

### FIELD OF THE INVENTION

The invention relates to the field of cardiac monitoring and more specifically to the field of portable cardiac monitoring.

### BACKGROUND

Cardiovascular disease (CVD) affects more than 80 million people as of 2008 and is the leading cause of death in the U.S. In 2008, costs associated with CVD were $297.7 billion, and by 2030, costs are expected to reach $1.117 trillion per year for CVD in the U.S. alone. To help reduce these costs, there is a push to change the current hospital-centric, reactive healthcare delivery system to one that focuses on early detection and diagnosis through extended, personalized monitoring.

Continuously monitoring vital signs such as heart rate (HR) and heart intervals can provide the data necessary for early diagnosis of CVD. What is needed is an inexpensive, wearable and portable monitor capable of measuring certain vital signs.

The present invention addresses this need.

### SUMMARY OF THE INVENTION

In one aspect, the invention relates to a physiological monitor for measuring a pulsatile motion signal (MoCG) that is delayed from, but at the same rate as, the heartbeat of a user. In one embodiment, the system includes a housing configured to be worn on a body of a user; at least one MoCG sensor, within the housing, that measures a pulsatile motion signal (MoCG) that is delayed from, but at the same rate as, the heartbeat of the user; and at least one data processor that calculates, solely based on an output of the at least one MoCG sensor, at least one of (i) heart rate (HR) and activity level for the user, and (ii) respiratory rate (RR), stroke volume (SV), and cardiac output (CO) for the user. In another embodiment, the at least one data processor is within the housing. In still another embodiment, the system includes at least one data transmitter coupled to the at least one MoCG sensor, wherein the at least one data processor is part of a remote computing system that receives data from the at least one data transmitter. In yet another embodiment, the remote computing system is selected from a group consisting of: mobile communications devices, wearable devices, mobile telephones, tablet computers, data collection devices, and network enabled medical devices. In still yet another embodiment, the housing is worn on an extremity of the user. In one embodiment, the housing is worn on or adjacent a bicep of the user. In another embodiment, the housing is on or adjacent a wrist of the user. In yet another embodiment, the housing is on or adjacent the torso of the user. In still yet another embodiment, the housing is on or adjacent a foot of the user. In still another embodiment, the housing is carried by the body of the user.

In one embodiment, the MoCG sensor includes one or more of an accelerometer and a gyroscope. In another embodiment, the system includes at least one optical sensor, within the housing, for measuring photoplethysmogram (PPG) of the user. In still another embodiment, at least one data processor calculates blood pressure (BP) based on a calculated time delay between a reference point in the MoCG and a reference point in the PPG. In yet another embodiment, the reference point is selected from a group consisting of a maxima, a minima, a point of maximum slope, or the midpoint of the maxima and minima of the signal. In still yet another embodiment, the at least one data processor calculates at least one of (i) HR and RR for the user, and (ii) blood oxygenation (SpO2) for the user, solely using the measured PPG. In one embodiment, the system further includes, within the housing, at least one circuit for measuring an electrocardiogram (ECG) of the user. In another embodiment, the at least one data processor calculates a pre-ejection period (PEP) in response to the delay between a peak in the ECG and a peak in the MoCG. In yet another embodiment, the at least one data processor calculates HR and RR from ECG.

In one embodiment, the system further includes at least one optical sensor for measuring a PPG, and wherein at least one data processor calculates at least three of: HR, BP, RR, SV, CO, activity level, SpO2, and PEP for the user based on the measured ECG and the measured PPG for the user. In another embodiment, the system includes, within the housing, memory for storing data and a transmitter that transmits data to at least one remote computing device. In still another embodiment, the system further includes a module for providing sensory feedback to the user upon the occurrence of at least one calculated event. In yet another embodiment, the system includes a module for providing sensory feedback to the user upon user request.

In one embodiment, the system includes a housing configured to be worn on a body of a user; at least one MoCG sensor, within the housing, that measures a pulsatile motion signal (MoCG) that is delayed from, but at the same rate as, the heartbeat of the user; and at least one optical sensor, within the housing, for measuring photoplethysmogram (PPG) of the user. In another embodiment, the system includes at least one data processor, wherein the at least one data processor calculates, solely based on an output of the at least one MoCG sensor, at least one of (i) heart rate (HR) and activity level for the user and (ii) respiratory rate (RR), stroke volume (SV), and cardiac output (CO) for the user. In still another embodiment, the system further includes at least one data transmitter coupled to the at least one MoCG sensor and the at least one optical sensor, and wherein at least one data processor is part of a remote computing system that receives data from at least one data transmitter. In yet still another embodiment, the remote computing system is selected from a group consisting of: mobile communications devices, wearable devices, mobile telephones, tablet computers, data collection devices, and network enabled medical devices.

In one embodiment, the at least one data processor calculates blood pressure (BP) based on a calculated time delay between a reference point in the MoCG and a reference point in the PPG. In another embodiment, the at least one data processor calculates at least one of (i) HR, RR for the user, and (ii) blood oxygenation (SpO2) for the user solely using the measured PPG. In another embodiment, the system includes, within the housing, at least one circuit for measuring an electrocardiogram (ECG) of the user. In another embodiment, the at least one data processor calculates a pre-ejection period (PEP) in response to the delay between a peak in the ECG and a peak in the MoCG. In yet another embodiment, the at least one data processor calculates HR and RR from ECG. In yet another embodiment, the system further includes, within the housing, memory for storing data and a transmitter that transmits data to at least one remote computing device. In still yet another embodiment, the system further includes a module for providing sensory feedback to the user upon the occurrence of at least one calculated event. In another embodiment, the system further includes a module for providing sensory feedback to the user upon user request.

In one embodiment, the system includes at least one data processor and a memory, storing instructions, which when executed by the at least one data processor, result in operations including receiving data from a first sensor characterizing pulsatile motion in the body (MoCG) of a user, the first sensor being part of a monitor worn on a body of the user; calculating, solely based on the received data, heartbeat related parameters for the user comprising at least one of (i) heart rate (HR) and activity level for the user, and (ii) respiratory rate (RR), stroke volume (SV), and cardiac output (CO) for the user and providing data characterizing the heartbeat related parameters. In another embodiment, the providing of data includes one or more of displaying at least a portion of the data characterizing the heartbeat related parameters, transmitting at least a portion of the data characterizing the heartbeat related parameters to a remote computing device, loading at least a portion of the data characterizing the heartbeat related parameters into memory, and storing at least a portion of the data characterizing the heartbeat related parameters into a data storage device. In yet another embodiment, the operations further include receiving data from at least one optical sensor for measuring photoplethysmogram (PPG) of the user, the at least one optical sensor being part of the monitor worn on the body of the user, and calculating blood pressure (BP) based on a calculated time delay between a reference point in the MoCG and a reference point in the PPG, and providing data characterizing the calculated blood pressure. In still yet another embodiment, the operations further comprise calculating at least one of (i) HR and RR for the user, and (ii) blood oxygenation (SpO2) for the user, solely using the measured PPG. In one embodiment, the operations further include receiving data from at least one electrocardiogram (ECG) sensor for measuring ECG of the user, the at least one ECG sensor being part of the monitor worn on the body of the user, and calculating at least three of: HR, RR, SV, CO, activity level, SpO2, and PEP for the user in response to the MoCG, ECG and the PPG.

In another aspect, the invention relates to a method including the steps of receiving data from a first sensor characterizing pulsatile motion in the body (MoCG) of a user, the first sensor being part of a monitor worn on the body of the user; calculating, solely based on the received data, heartbeat related parameters for the user comprising at least one of (i) heart rate (HR) and activity level for the user, and (ii) respiratory rate (RR), stroke volume (SV), and cardiac output (CO) for the user; and providing data characterizing the heartbeat related parameters. In one embodiment, the step of providing data includes one or more of displaying at least a portion of the data characterizing the heartbeat related parameters, transmitting at least a portion of the data characterizing the heartbeat related parameters to a remote computing device, loading at least a portion of the data characterizing the heartbeat related parameters into memory, and storing at least a portion of the data characterizing the heartbeat related parameters into a data storage device. In another embodiment, the method further includes the steps of receiving data from at least one optical sensor for measuring photoplethysmogram (PPG) of the user, the at least one optical sensor being part of the monitor worn on the body of the user; and calculating blood pressure (BP) based on a calculated time delay between a reference point in the MoCG and a reference point in the PPG; and providing data characterizing the calculated blood pressure.

In one embodiment, the method further includes the step of calculating at least one of (i) HR and RR for the user and (ii) blood oxygenation (SpO2) for the user solely using the measured PPG. In another embodiment, the method further includes receiving data from at least one electrocardiogram (ECG) sensor for measuring ECG of the user, the at least one ECG sensor being part of the monitor worn on the body of the user; and calculating at least three of: HR, RR, SV, CO, activity level, SpO2, and PEP for the user in response to the MoCG, ECG and the PPG.

In another aspect, the invention relates to a non-transitory computer program product. In one embodiment, the product includes stored instructions, which when executed by at least one data processor of at least one computing system, results in operations including receiving data from a first sensor characterizing pulsatile motion in the body (MoCG) of a user, the first sensor being part of a monitor worn on the body of the user; calculating, solely based on the received data, heartbeat related parameters for the user comprising at least one of: (i) heart rate (HR) and activity level for the user, and (ii) respiratory rate (RR), stroke volume (SV) and cardiac output (CO) for the user; and providing data characterizing the heartbeat related parameters. In another embodiment, the steps of providing data include one or more of displaying at least a portion of the data characterizing the heartbeat related parameters, transmitting at least a portion of the data characterizing the heartbeat related parameters to a remote computing device, loading at least a portion of the data characterizing the heartbeat related parameters into memory, and storing at least a portion of the data characterizing the heartbeat related parameters into a data storage device. In yet another embodiment, the operations further include receiving data from at least one optical sensor for measuring photoplethysmogram (PPG) of the user, the at least one optical sensor being part of the monitor worn on the body of the user; and calculating blood pressure (BP) based on a calculated time delay between a reference point in the MoCG and a reference point in the PPG; and providing data characterizing the calculated blood pressure. In still yet another embodiment, the operations further include: calculating at least one of (i) HR and RR for the user, and (ii) blood oxygenation (SpO2) for the user solely using the measured PPG. In another embodiment, the operations further include receiving data from at least one electrocardiogram (ECG) sensor for measuring ECG of the user, the at least one ECG sensor being part of the monitor worn on the body of the user; and calculating at least three of: HR, RR, SV, CO, activity level, SpO2, and PEP for the user in response to the MoCG, ECG and the PPG.

### BRIEF DESCRIPTION OF THE DRAWINGS

FIG. 1(a) is a block diagram of an embodiment of the system of the invention;

FIG. 1(b) is a block diagram of another embodiment of the system of the invention;

FIG. 2(a) is a block diagram of an embodiment of the ECG measuring module shown in FIG. 1(a);

FIG. 2(b) is a block diagram of an embodiment of the PPG measuring module shown in FIG. 1(a);

FIGS. 3 (a)-(c) are a series of graphs showing the ECG, MoCG and PPG signals measured by the system of FIG. 1(a);

FIGS. 4(a) and (b) are graphs of blood pressure measured using a cuff and determined by an algorithm using the measured physiologic parameters by the system of FIG. 1(a);

FIG. 5 is a graph of a PPG signal, the filtered signal, and the extracted respirations as measured by the system of FIG. 1(a); and

FIG. 6(a-d) are drawings of various locations at which the device may be carried.

### DESCRIPTION OF AN EMBODIMENT OF THE INVENTION

This invention relates to a wearable device that measures a pulsatile motion signal of the body. This pulsatile signal, which is measurable by an accelerometer or a gyroscope, is the result of a mechanical motion of portions of the body that occurs in response to blood being pumped during a heartbeat. This motion is a direct manifestation of Newton's Third Law, where the internal flow of blood causes a mechanical reaction that is externally measurable. As a result, this motion cardiogram signal (denoted as “MoCG”) corresponds to, but is delayed from, the heartbeat.

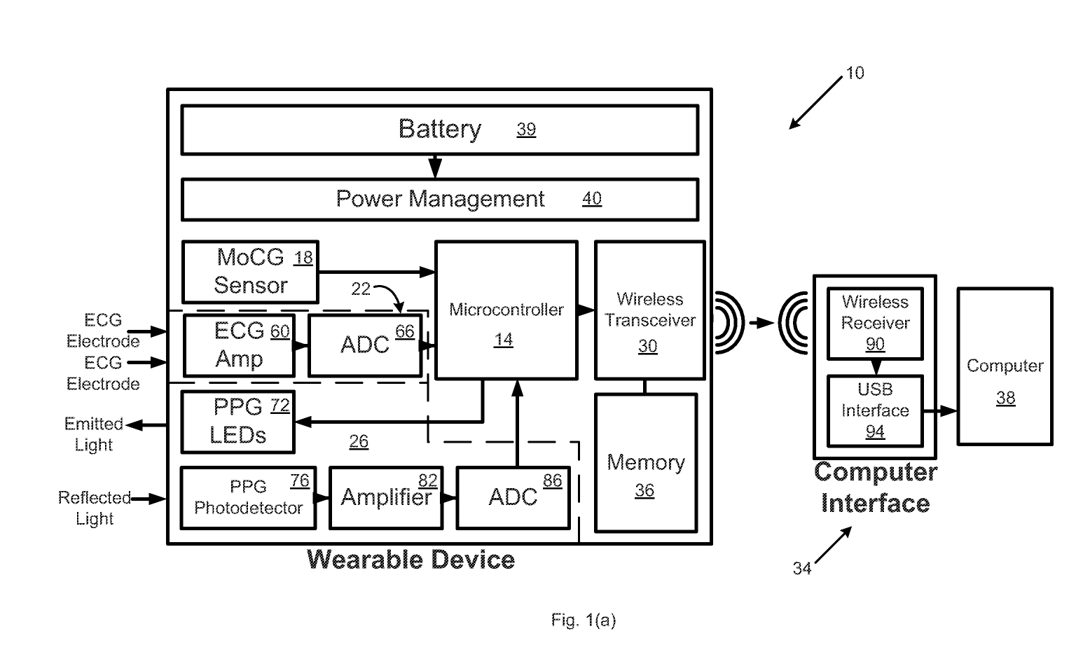

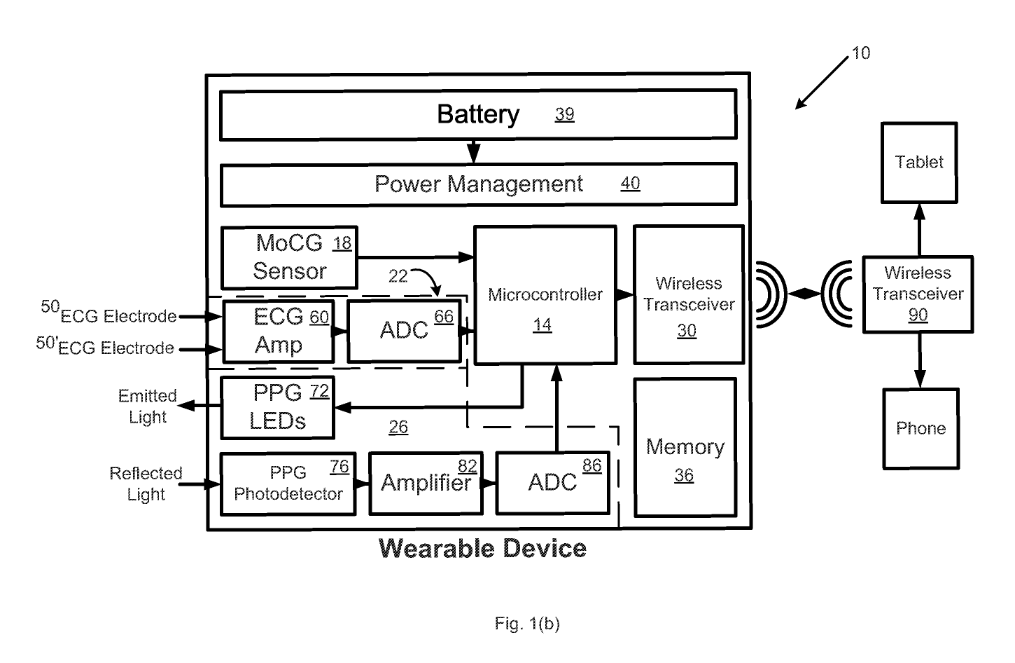

Referring to FIG. 1, and in brief overview, an embodiment of a wearable heart monitor 10 includes a microcontroller 14 having an input in communication with an MoCG accelerometer 18, an electrocardiogram (ECG) module 22, and a photoplethysmogram (PPG) module 26. The output of the microcontroller 14 is in communication with a wireless transceiver 30, that transmits the microcontroller output to a computer interface transceiver 34 that is the front end to a computer 38, running analytic software. Alternatively, the data may be stored in optional memory 36 and retrieved at a later time. The microcontroller 14 and related modules 18, 22, 26, 30, 36 are powered by a 3V battery 39 through a power management module 40 that includes 2.5V linear regulator and a 2.7V switching regulator. Thus, the present device can measure MoCG, PPG, and ECG simultaneously and continuously, and can be used to measure or calculate HR, BP, RR, SV, CO, activity level, SpO2, and PEP.

In operation, the MoCG sensor 18, the ECG module 22, and the PPG module 26 transmit signals to the microcontroller 14 indicating body motion, ECG, and PPG, respectively, and the microcontroller 14 transmits those signals through a wireless transmitter 30 to the computer interface receiver 34 for analysis by the computer 38. In an alternative embodiment, the wireless transmitter communicates over a cell phone network to a distant computer. In another embodiment, the microcontroller 14 stores the data in memory 36 rather than sending the data wirelessly. Periodically the memory 36 can be interrogated by a computer temporarily attached to the device and the data removed and analyzed. In an alternate embodiment, the data is analyzed by the microprocessor 14 and only the results are transmitted to the computer 38. The device in one embodiment has a visual or auditory feedback to the user in the case of alarm or data request by the user. FIG. 1(b) is a diagram of the system of FIG. 1(a), but depicting that the data is analyzed by the microprocessor 14 and only the results are transmitted to a mobile device such as a tablet or smartphone rather than a computer.

Considering each component in more detail, MoCG is measured using a motion sensor which in various embodiments is an accelerometer and/or a gyroscope 18. In one embodiment, a Bosch Sensortec Ltd. (Kusterdingen, Germany) BMA180 MEMS triaxial accelerometer with 10 Hz bandwidth, 14 bit resolution, 0.69 mG_{RMS} of noise, ±2G range, and integrated digital output or equivalent is used. The integrated digital output of the accelerometer/gyroscope 18 is input through a serial port on the microcontroller 14. In one embodiment, the microcontroller 14 is an MSP430 16-bit ultra-low power microcontroller (Texas Instruments Incorporated, Dallas, Tex.).

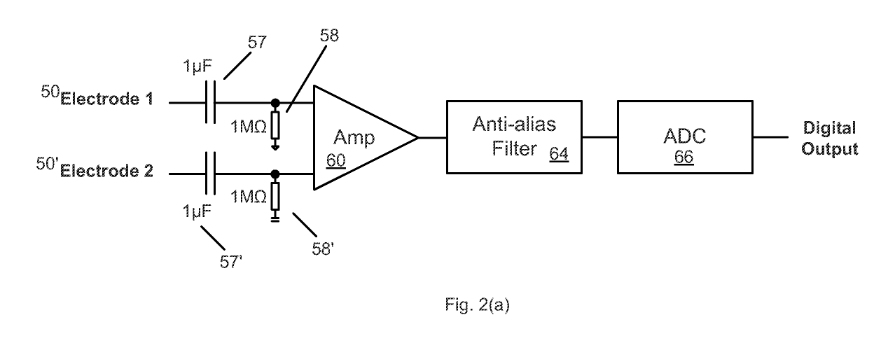

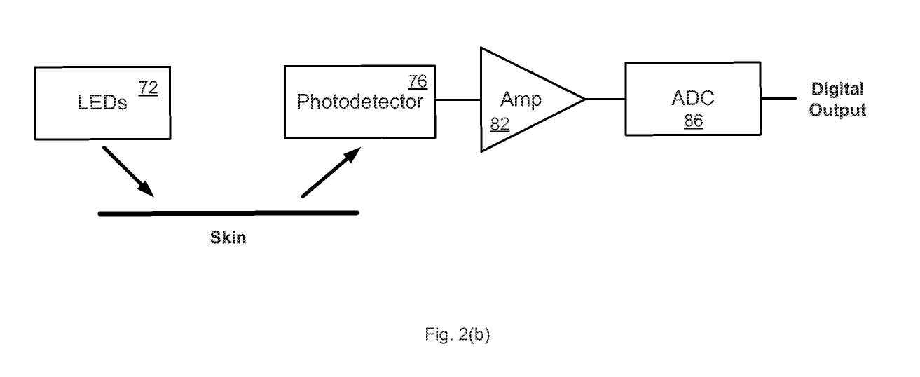

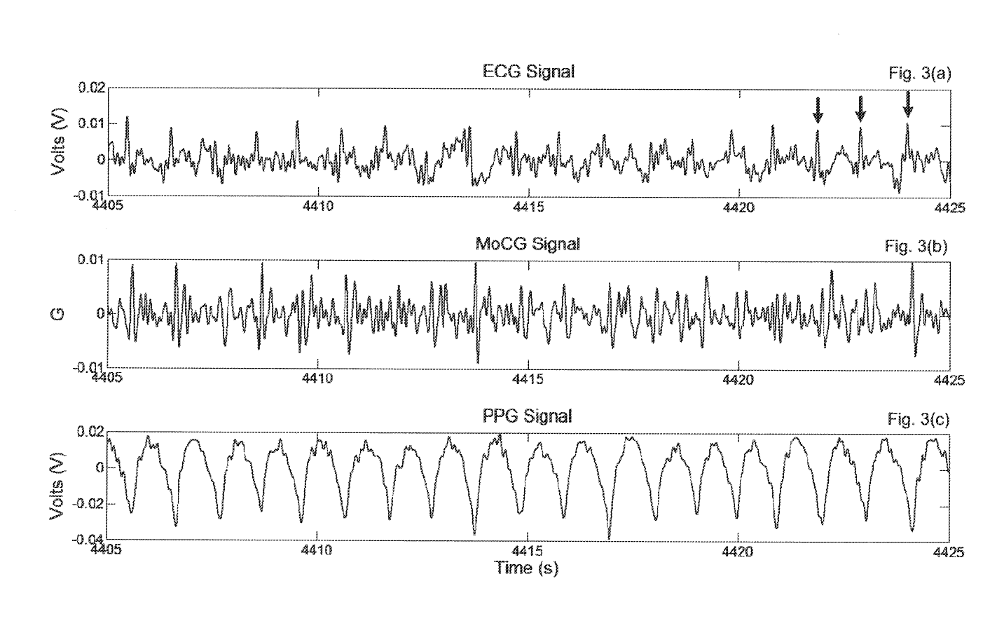

Referring to FIG. 2(a), the ECG module 22 includes two input terminals, each for connection to a respective ECG gel electrode 50, 50′. The input terminals transmit the signals from the electrodes to two inputs of an amplifier 60 through a respective filter 56, 56′. Each filter includes a capacitor 57, 57′ (generally 57) connected in series between its respective electrode 50, 50′ (generally 50) and the respective input terminal of the amplifier 60, and a resistor 58, 58′ connected between the respective input terminal of the amplifier 60 and ground. The output of the amplifier 60 is the input to an anti-alias filter 64. The output of the anti-alias filter 64 in turn is the input to a 12-bit ADC 66 operating at 155 Hz. The resulting digital output is also an input to the microcontroller 14 through a serial port. In one embodiment, the ECG front-end uses a low noise instrumentation amplifier (INA333) (Texas Instruments, Dallas, Tex.) and a 12-bit analog-to-digital converter (AD7466) (Analog Devices, Norwood, Mass.) to amplify and digitize the single-lead ECG from two gel electrodes.

Referring also to FIG. 2(b), the PPG module includes LEDs 72 whose output is controlled by the microcontroller 14. Light from the LEDs 72 is directed toward the skin of a patient, and the reflected light is modulated by blood flow in the region of skin. Light reflected by the body is received by a photodetector 76 and the resulting signal amplified by amplifier 82 before being converted to a digital signal by a 12-bit ADC 86 that is an input to a serial port of the microcontroller 14. In one embodiment, the PPG module uses an infrared LED and the photodetector package EE-SY193 (Omron Electronic Components LLC, Schaumburg Ill.). The signal from the photodetector is amplified by an amplifier OPA333 (Texas Instruments Incorporated, Dallas, Tex.) and is digitized using a 12-bit analog-to-digital converter (AD7466) (Analog Devices, Norwood, Mass.) and the resulting values transmitted to the microcontroller 14 over a serial port.

The computer interface receiver 34 includes a wireless receiver 90 connected to a USB interface 94 that transmits the received signal to the computer 38 for analysis. In various embodiments, the computer 38 is a laptop, a server, a tablet, a smartphone or other computing device. In one embodiment, the analysis software is MATLAB (The MathWorks, Inc., Natick, Mass.)

An example measurement of MoCG, PPG, and ECG signals as measured by the described system is shown in FIG. 3. FIG. 3(a) is a time series of an ECG signal measured by the system. FIG. 3(b) is a time series of an MoCG signal measured by the system measured at the same time as FIG. 3(a). FIG. 3(c) is a time series of a PPG signal measured by the system at the same time as the signals in FIGS. 3(a) and (b).

In operation, the heart rate (HR) is obtainable from each of the ECG, PPG, and the MoCG signal because the MoCG signal corresponds to, but is delayed from, the heartbeat. The signal corresponding to the heart rate is visible in the 1-10 Hz range of MoCG signal. Further, because respiration also induces movement of the body, the MoCG signal itself contains a respiration signal. The respiration signal is visible in the 0-1 Hz range of MoCG signal. The amplitude of MoCG signal relates to the stroke volume (SV) of the heart, as the amount of blood pumped internally causes the body's pulsatile vibration. SV can be calculated from the MoCG pulsatile peaks' amplitudes using SV=C*(MoCG peak amplitude)+D, where C and D are constants obtained from calibration. The product of HR and SV is the cardiac output (CO). Activity level, defined as motion data that ranges above 50 mG of acceleration, is directly measured as large scale motions (i.e. >50 mG) sensed by the MoCG sensor.

When the MoCG data are paired with the photoplethysmogram (PPG) data, additional measurements are derivable. The time delay (denoted as “MPTT”) measured between a reference point of MoCG and a reference point on the PPG is an indication of blood pulse transit time. The reference point, such as a maxima, a minima, a point of maximum slope or the midpoint of the maxima and minima of the signal can be used. The MPTT is related to blood pressure (BP) via the following equation based on the Moens-Korteweg and Hughes equations based on fluid dynamics:

BP=(A*ln(MPTT))+B+P_{hydro} (1)

where (BP) is blood pressure, and A and B are constants that are derived from calibration. In one embodiment, calibration includes measuring two different MPTTs at two different BPs on the same user, thus solving for the two unknowns A and B. A and B may depend on parameters such as arterial length, arterial radius, arterial wall thickness, arterial elasticity, and blood density. As a result, this device enables single-site cuffless BP measurement where all sensors are at a single location. P_{hydro} is a hydrostatic component that may be present and is dependent on the height of the sensor location relative to the location of the heart of the wearer. As a result, P_{hydro} is dependent on the placement of the sensor and the orientation and position of wearer.

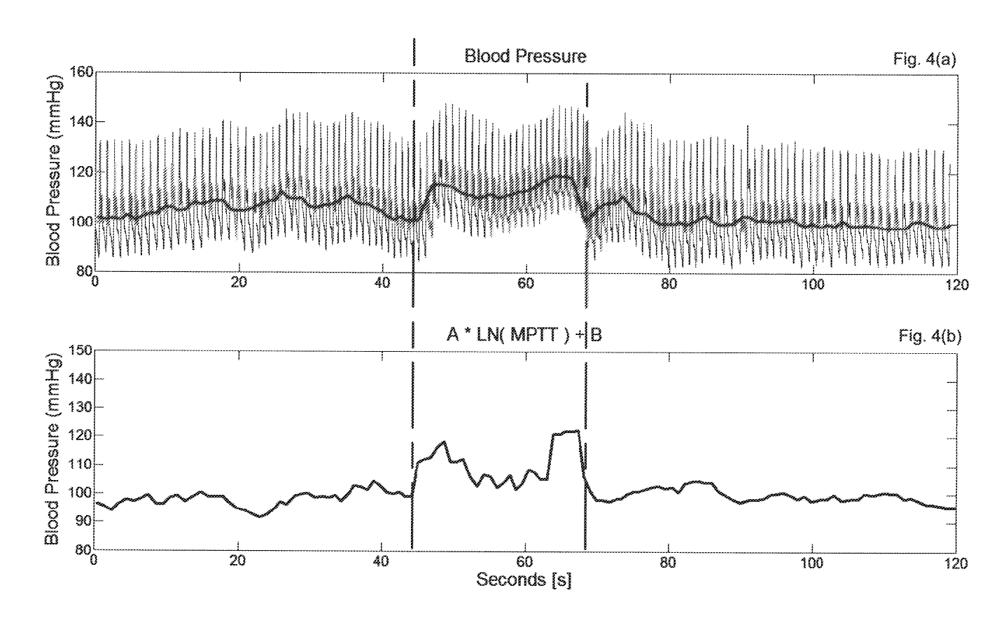

An example of the result of a calculation of BP from MoCG and PPG is shown in FIG. 4. FIG. 4(a) is an actual BP measurement for reference. FIG. 4(b) is a measurement of BP as measured by the device using equation (1) in which the P_{hydro} has been ignored.

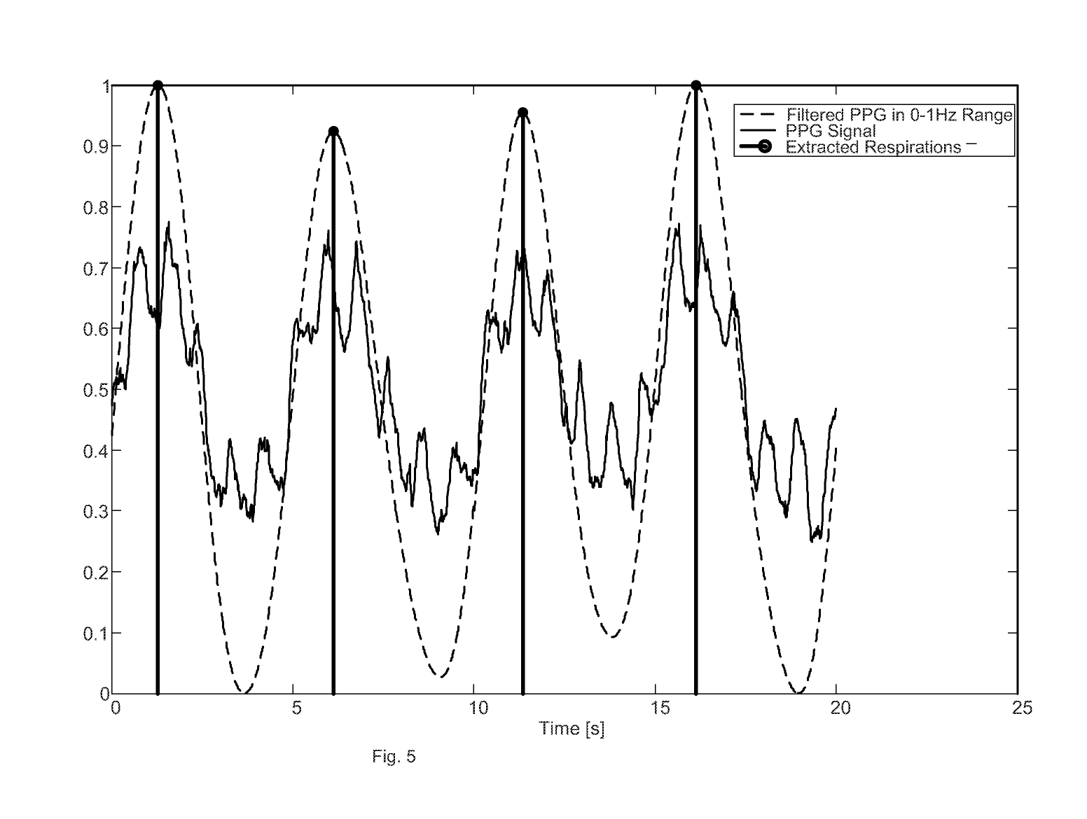

Also, PPG by itself is a pulsatile signal synchronized with the heartbeat and can be used to determine heart rate (HR). The heart rate signal can be visible in the 1-10 Hz range of PPG as shown in FIG. 5. Further, the baseline of PPG is modulated by respiration. The respiration signal can be visible in the 0-1 Hz range of PPG. When more than one color is used for the LED of the PPG module, blood oxygenation (SpO₂) can be obtained using the pulse oximetry theory.

The pre-ejection period is defined as the time between the peak of ECG (R-wave) and the ejection of blood from the heart. Because the MoCG's peak occurs soon after the ejection of blood from the heart, the time delay from the peak of the ECG to the peak of the MoCG can be used to calculate the heart's pre-ejection period. Also, the ECG by itself is a pulsatile signal synchronized with the heartbeat and can be used directly to measure HR. The heart rate signal can be visible (see exemplary arrows) in the 1-50 Hz range of ECG (FIG. 3(a)).

Additional parameters are also obtainable from the ECG. For example, the ECG peak amplitudes are modulated by respiration. Therefore, the frequency of oscillation of the ECG peak amplitudes is the RR.

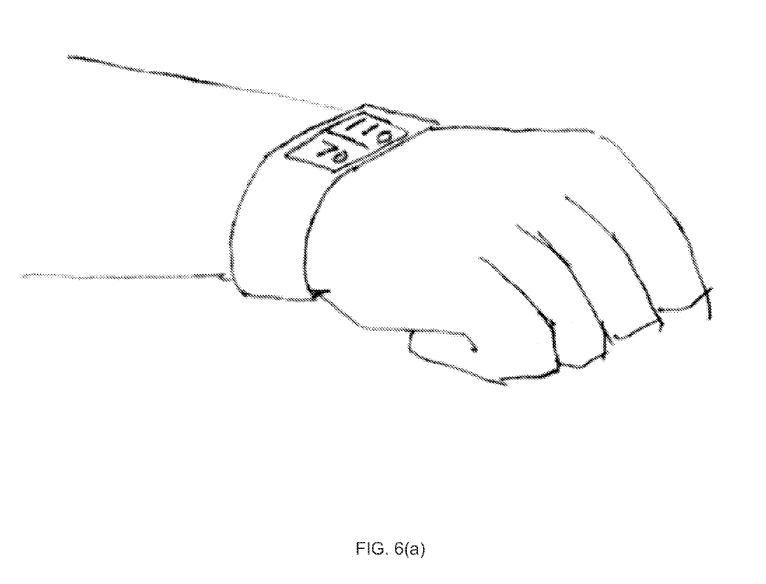

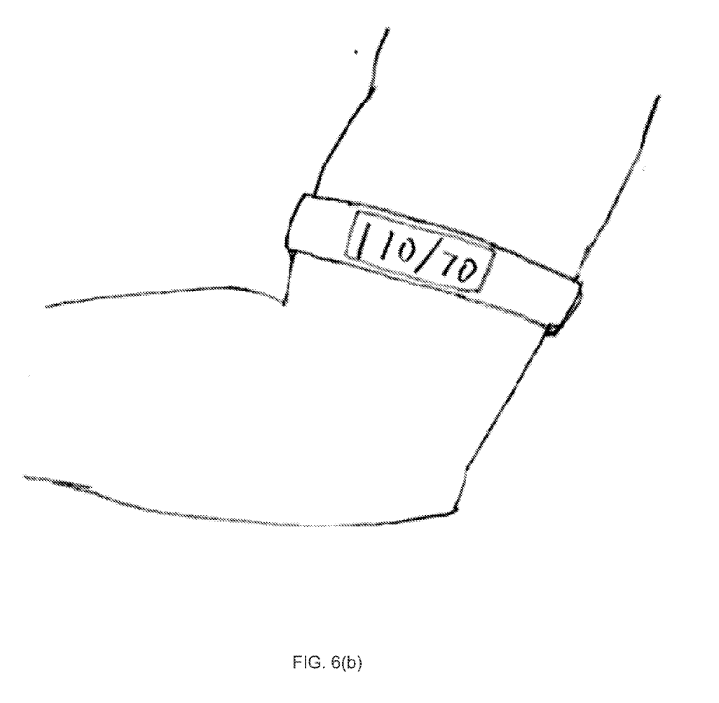

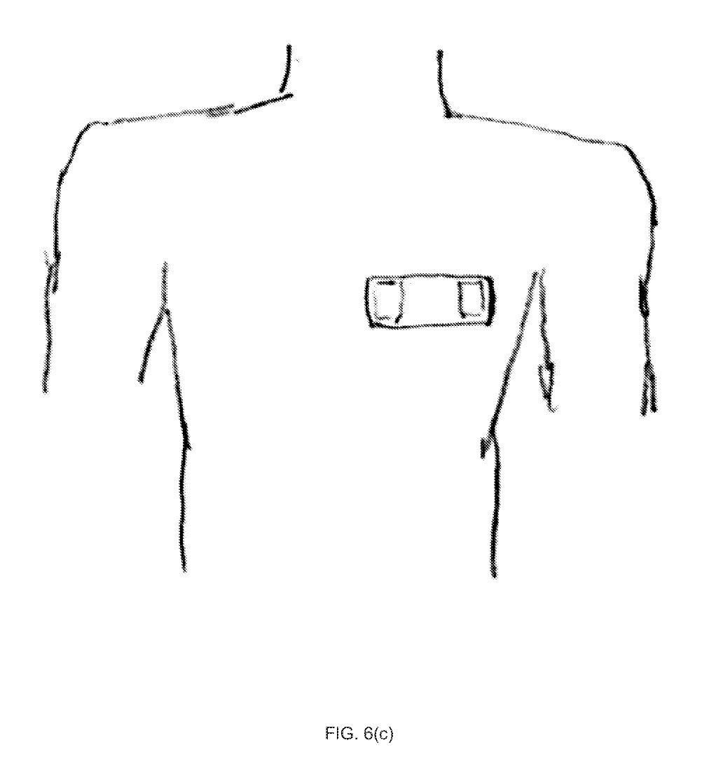

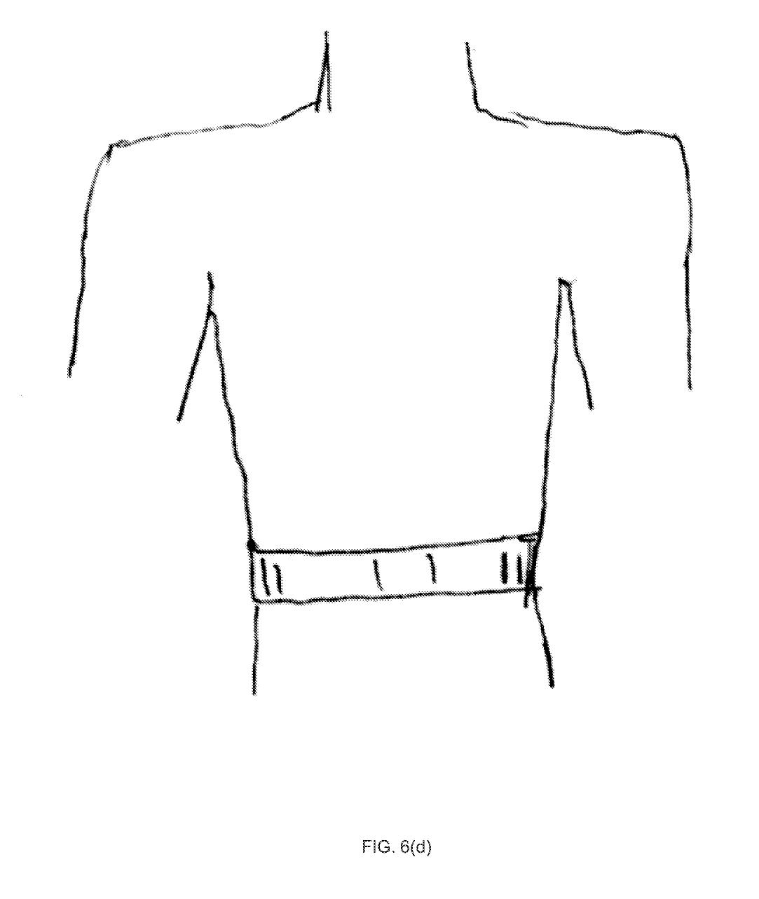

Because the MoCG signal is the result of mechanical motion that arises from arterial blood flow, this device is wearable anywhere on the body, making MoCG measurements either directly (such as by an armband, wristband, chest patch, undergarment) or indirectly (such as implemented as part of a smartphone inside one's pocket). The wrist location (FIG. 6a)) is convenient for the user and has high quality PPG but the MoCG is more easily corrupted by motion artifacts from hand movements. The bicep location (FIG. 6(b)) has high quality MoCG but PPG is diminished, and P_(hydro) is negligible at this location, thus leading to simplified BP calculations. The torso location (FIG. 6(c)) has less motion artifacts but is less convenient for the user to wear on a daily basis unless it is integrated into a belt or undergarment of the user (FIG. 6(d)). The foot location has significant motion artifacts but can be an easier location to track activity level arising from walking or running.

It should be understood that the order of steps or order for performing certain actions is immaterial so long as the present teachings remain operable. Moreover, two or more steps or actions may be conducted simultaneously.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes and not as construction drawings. Omitted details and modifications or alternative embodiments are within the purview of persons of ordinary skill in the art.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

One or more aspects or features of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system, including at least one programmable processor which may be special or general purpose, coupled to receive data and instructions from, and to transmit data and instructions to, a storage system, at least one input device (e.g., mouse, touch screen, etc.), and at least one output device.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as, for example, would a non-transient solid state memory or a magnetic hard drive or any equivalent storage medium. The machine-readable medium can alternatively or additionally store such machine instructions in a transient manner, such as, for example, would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor, for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The subject matter described herein may be implemented in a computing system that includes a back-end component (e.g., a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow(s) depicted in the accompanying figures and/or described herein do not necessarily require the particular order shown, or sequential order, to achieve desirable results. Other implementations may be within the scope of the following claims.

What is claimed is:

1. A system comprising:
a housing configured to be worn on a body of a user;
at least one MoCG sensor, within the housing, that measures a pulsatile motion signal (MoCG) that is delayed from, but at the same rate as, the heartbeat of the user; and
at least one data processor that calculates, solely based on an output of the at least one MoCG sensor, at least one of (i) heart rate (HR) and activity level for the user, and (ii) respiratory rate (RR), stroke volume (SV), and cardiac output (CO) for the user.

2. The system of claim 1, wherein the at least one data processor is within the housing.

3. The system of claim 1 further comprising: at least one data transmitter coupled to the at least one MoCG sensor, and wherein the at least one data processor is part of a remote computing system that receives data from the at least one data transmitter.

4. The system of claim 3, wherein the remote computing system is selected from a group consisting of: mobile communications devices, wearable devices, mobile telephones, tablet computers, data collection devices, and network enabled medical devices.

5. The system of claim 1, wherein the housing is worn on an extremity of the user.

6. The system of claim 1, wherein the housing is worn on or adjacent a bicep of the user.

7. The system of claim 1, wherein the housing is on or adjacent a wrist of the user.

8. The system of claim 1, wherein the housing is on or adjacent the torso of the user.

9. The system of claim 1, wherein the housing is on or adjacent a foot of the user.

10. The system of claim 1, wherein the housing is carried by the body of the user.

11. The system of claim 1, wherein the MoCG sensor comprises one or more of an accelerometer and a gyroscope.

12. The system of claim 1 further comprising at least one optical sensor, within the housing, for measuring photoplethysmogram (PPG) of the user.

13. The system of claim 12, wherein at least one data processor calculates blood pressure (BP) based on a calculated time delay between a reference point in the MoCG and a reference point in the PPG.

14. The system of claim 13, wherein the reference point is selected from a group consisting of: a maxima, a minima, a point of maximum slope, or the midpoint of the maxima and minima of the MoCG and the PPG signals.

15. The system of claim 13, wherein the at least one data processor calculates at least one of (i) HR and RR for the user, and (ii) blood oxygenation (SpO2) for the user, solely using the measured PPG.

16. The system of claim 1 further comprising, within the housing, at least one circuit for measuring an electrocardiogram (ECG) of the user.

17. The system of claim 16, wherein the at least one data processor calculates pre-ejection period (PEP) in response to the delay between a peak in the ECG and a peak in the MoCG.

18. The system of claim 16, wherein the at least one data processor calculates HR and RR from ECG.

19. The system of claim 16 further comprising at least one optical sensor for measuring a PPG, and wherein at least one data processor calculates at least three of: HR, BP, RR, SV, CO, activity level, SpO2, and PEP for the user based on the measured ECG and the measured PPG for the user.

20. The system of claim 1 further comprising, within the housing, memory for storing data and a transmitter that transmits data to at least one remote computing device.

21. The system of claim 1 further comprising a module for providing sensory feedback to the user upon the occurrence of at least one calculated event.

22. The system of claim 1 further comprising a module for providing sensory feedback to the user upon user request.

23. A system comprising:
a housing configured to be worn on a body of a user;
at least one MoCG sensor, within the housing, that measures a pulsatile motion signal (MoCG) that is delayed from, but at the same rate as, the heartbeat of the user; and
at least one optical sensor, within the housing, for measuring photoplethysmogram (PPG) of the user.

24. A system as in claim 23, further comprising at least one data processor.

25. The system of claim 24, wherein the at least one data processor calculates, solely based on an output of the at least one MoCG sensor, at least one of (i) heart rate (HR) and activity level for the user and (ii) respiratory rate (RR), stroke volume (SV), and cardiac output (CO) for the user.

26. The system of claim 24, wherein the at least one data processor is within the housing.

27. The system of claim 24 further comprising: at least one data transmitter coupled to the at least one MoCG sensor and the at least one optical sensor, and wherein at least one data processor is part of a remote computing system that receives data from at least one data transceiver.

28. The system of claim 27, wherein the remote computing system is selected from a group consisting of: mobile communications devices, wearable devices, mobile telephones, tablet computers, data collection devices, and network enabled medical devices.

29. The system of claim 23, wherein the housing is worn on an extremity of the user.

30. The system of claim 23, wherein the housing is worn on or adjacent a bicep of the user.

31. The system of claim 23, wherein the housing is on or adjacent a wrist of the user.

32. The system of claim 23, wherein the housing is on or adjacent the torso of the user.

33. The system of claim 23, wherein the housing is on or adjacent a foot of the user.

34. The system of claim 23, wherein the housing is carried by the body of the user.

35. The system of claim 23, wherein the MoCG sensor comprises one or more of an accelerometer and a gyroscope.

36. The system of claim 24, wherein the at least one data processor calculates blood pressure (BP) based on a calculated time delay between a reference point in the MoCG and a reference point in the PPG.

37. The system of claim 36, wherein the reference point is selected from a group consisting of: a maxima, a minima, a point of maximum slope, or the midpoint of the maxima and minima of the MoCG and the PPG signals.

38. The system of claim 24, wherein the at least one data processor calculates at least one of (i) HR, RR for the user, and (ii) blood oxygenation (SpO2) for the user solely using the measured PPG.

39. The system of claim 23 further comprising, within the housing, at least one circuit for measuring an electrocardiogram (ECG) of the user.

40. The system of claim 39, wherein the at least one data processor calculates pre-ejection period (PEP) in response to the delay between a peak in the ECG and a peak in the MoCG.

41. The system of claim 39, wherein the at least one data processor calculates HR and RR from ECG.

42. The system of claim 23 further comprising, within the housing, memory for storing data and a transceiver that transmits data to at least one remote computing device.

43. The system of claim 23 further comprising a module for providing sensory feedback to the user upon the occurrence of at least one calculated event.

44. The system of claim 23 further comprising a module for providing sensory feedback to the user upon user request.

45. A method comprising:
receiving data from a first sensor characterizing pulsatile motion in the body (MoCG) of a user, the first sensor being part of a monitor worn on a body of the user;
calculating, solely based on the received data, heartbeat related parameters for the user comprising at least one of (i) heart rate (HR) and activity level for the user, and (ii) respiratory rate (RR), stroke volume (SV), and cardiac output (CO) for the user; and
providing data characterizing the heartbeat related parameters.

46. The method as in claim 45, wherein providing data comprises one or more of: displaying at least a portion of the data characterizing the heartbeat related parameters, transmitting at least a portion of the data characterizing the heartbeat related parameters to a remote computing device, loading at least a portion of the data characterizing the heartbeat related parameters into memory, and storing at least a portion of the data characterizing the heartbeat related parameters into a data storage device.

47. The method of claim 45 further comprising:
receiving data from at least one optical sensor for measuring photoplethysmogram (PPG) of the user, the at least one optical sensor being part of the monitor worn on the body of the user; and
calculating blood pressure (BP) based on a calculated time delay between a reference point in the MoCG and a reference point in the PPG; and
providing data characterizing the calculated blood pressure.

48. The method of claim 47 further comprising calculating at least one of (i) HR and RR for the user and (ii) blood oxygenation (SpO2) for the user, solely using the measured PPG.

49. The method of claim 47 further comprising:
receiving data from at least one electrocardiogram (ECG) sensor for measuring ECG of the user, the at least one ECG sensor being part of the monitor worn on the body of the user; and
calculating at least three of: HR, RR, SV, CO, activity level, SpO2, and PEP for the user in response to the MoCG, ECG and the PPG.

50. A non-transitory computer program product storing instructions, which when executed by at least one data processor of at least one computing system, results in operations comprising:
receiving data from a first sensor characterizing pulsatile motion in the body (MoCG) of a user, the first sensor being part of a monitor worn on a body of the user;
calculating, solely based on the received data, heartbeat related parameters for the user comprising at least one of: (i) heart rate (HR) and activity level for the user, and (ii) respiratory rate (RR), stroke volume (SV) and cardiac output (CO) for the user; and
providing data characterizing the heartbeat related parameters.

51. The computer program product as in claim 50, wherein the providing of data comprises one or more of: displaying at least a portion of the data characterizing the heartbeat related parameters, transmitting at least a portion of the data characterizing the heartbeat related parameters to a remote computing device, loading at least a portion of the data characterizing the heartbeat related parameters into memory, and storing at least a portion of the data characterizing the heartbeat related parameters into a data storage device.

52. The computer program product of claim 50, wherein the operations further comprise:
receiving data from at least one optical sensor for measuring photoplethysmogram (PPG) of the user, the at least one optical sensor being part of the monitor worn on the body of the user;
calculating blood pressure (BP) based on a calculated time delay between a reference point in the MoCG and a reference point in the PPG; and
providing data characterizing the calculated blood pressure.

53. The computer program product of claim 52, wherein the operations further comprise:
calculating at least one of (i) HR and RR for the user, and (ii) blood oxygenation (SpO2) for the user solely using the measured PPG.

54. The computer program product of claim 52, wherein the operations further comprise:
receiving data from at least one electrocardiogram (ECG) sensor for measuring ECG of the user, the at least one ECG sensor being part of the monitor worn on the body of the user; and
calculating at least three of: HR, RR, SV, CO, activity level, SpO2, and PEP for the user in response to the MoCG, ECG and the PPG.

55. A system comprising:
at least one data processor; and
memory storing instructions, which when executed by at least one data processor, result in operations comprising:
receiving data from a first sensor characterizing pulsatile motion in the body (MoCG) of a user, the first sensor being part of a monitor worn on a body of the user;
calculating, solely based on the received data, heartbeat related parameters for the user comprising at least one of (i) heart rate (HR) and activity level for the user, and (ii) respiratory rate (RR), stroke volume (SV), and cardiac output (CO) for the user; and
providing data characterizing the heartbeat related parameters.

56. The system of claim 55, wherein providing data comprises one or more of: displaying at least a portion of the data characterizing the heartbeat related parameters, transmitting at least a portion of the data characterizing the heartbeat related parameters to a remote computing device, loading at least a portion of the data characterizing the heartbeat related parameters into memory, and storing at least a portion of the data characterizing the heartbeat related parameters into a data storage device.

57. The system of claim 55, wherein the operations further comprise:
receiving data from at least one optical sensor for measuring photoplethysmogram (PPG) of the user, at least one optical sensor being part of the monitor worn on the body of the user;
calculating blood pressure (BP) based on a calculated time delay between a reference point in the MoCG and a reference point in the PPG; and
providing data characterizing the calculated blood pressure.

58. The system of claim 52, wherein the operations further comprise: calculating at least one of (i) HR and RR for the user, and (ii) blood oxygenation (SpO2) for the user solely using the measured PPG.

59. The system of claim 58, wherein the operations further comprise:
receiving data from at least one electrocardiogram (ECG) sensor for measuring ECG of the user, at least one ECG sensor being part of the monitor worn on the body of the user; and
calculating at least three of: HR, RR, SV, CO, activity level, SpO2, and PEP for the user in response to the MoCG, ECG and the PPG.
